(19) United States
(12) Patent Application Publication
(10) Pub. No.: US 2004/0089299 A1
(43) Pub. Date: May 13, 2004

(54) Inhaler

(76) Inventors: Stanley George Bonney, Ware (GB); Michael Birsha Davies, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,610

(22) PCT Filed: Sep. 26, 2001
(86) PCT No.: PCT/EP01/11097

(30) Foreign Application Priority Data
Oct. 20, 2000 (GB) 0025749.3

(51) Int. Cl. A61M 15/00; A61M 16/00

(52) U.S. Cl. 128/203.15; 128/203.19

(57) ABSTRACT

An inhaler for delivery of a dry powder medicament is disclosed that includes a breath sensor for sensing the breath of a patient, a reservoir for the dry powder, a meter for metering an amount of dry powder from the reservoir, and electro-mechanical coupling means for actuating said meter, wherein said coupling means is directly or indirectly responsive to said breath sensor.

### TECHNICAL DESCRIPTION

This invention relates to an inhaler for dispensing dry powder medicaments. In particular, the invention relates to an inhaler that does not require manual actuation by a patient.

### BACKGROUND TO THE INVENTION

Medical dispensers are well known for the dispensing of various kinds of medicament. Inhalation devices, such as metered dose inhalers (MDI) and dry powder inhalers are known for the delivery of medicament for the treatment of respiratory disorders such as asthma and chronic inflammatory pulmonary disease.

There are a number of different dry powder inhalers presently available. In one instance, the drug is encapsulated in hard gelatine and the inhaler comprises a device for perforating a capsule prior to the patient inhaling the contents. After the patient manually activates the opening of the capsule, a cloud of dry particles is directed into the nose or mouth of the patient usually by a channelling device such as a cylinder or open-ended cone. Concurrently with the release of the capsule contents, the patient inhales the drug particles into the lungs or nasal cavity. The vacuum created by the patient on inhalation is intended to empty the capsule contents.

The inhaler exemplified in EP-A-467172 accommodates a blister pack wherein each blister retains a dose of medicament in dry powder form. When a blister is positioned for dosing, a mechanism within the inhaler punctures the blister, releasing the contents for inhalation by the user as described supra.

U.S. Pat. No. 4,805,811 discloses a dry powder inhaler comprising a dry powder reservoir from which a dosing plate having a number of dosing “cups” is filled from the reservoir prior to inhalation. As with the examples described supra, this device requires manual metering and/or releasing of a metered dose prior to inhalation.

It may be understood that effective delivery of medicament to the patient using an inhalation device as described is to an extent dependent on the patient's ability to manually actuate the device (e.g. puncturing of a capsule) and to coordinate the actuation thereof with the taking of a sufficiently strong inward breath. For some patients, particularly young children, the elderly and the arthritic, manual actuation of the device can present difficulties. Other patients find it difficult to co-ordinate the taking of a reliable inward breath with actuation of the device. Both of these sets of patients run the risk that they do not receive the appropriate dose of medicament.

U.S. Pat. No. 5,239,992 discloses a loose powder inhaler wherein the vacuum created on inhalation by the user drives a dosing piston to measure and liberate a dose concurrent with inhalation of the drug. However, this device is reliant on the patient being able to draw sufficient breath to create the necessary vacuum and therefore does not alleviate the problems discussed supra.

The Applicants have now developed a dry powder inhaler that does not require manual actuation by the patient.

### SUMMARY OF THE INVENTION

Accordingly, in one aspect, the invention provides an inhaler for delivery of a dry powder medicament, the inhaler comprising: a breath sensor for sensing the breath of a patient; a reservoir for said dry powder; a meter for metering an amount of dry powder from said reservoir; and electromechanical coupling means for actuating said meter, wherein said coupling means is directly or indirectly responsive to said breath sensor.

Metering of the medicament is wholly dependent on the actuation of the breath sensor by the patient's breath. Accordingly, the medicament is protected from unintentional manual actuation of the dispenser whereby the dose may be lost or exposed to the environment.

Metering of the dry powder medicament immediately prior to inhalation has a number of advantages. Firstly, the medicament has no time to absorb moisture from its environment outside the dry powder reservoir. Also, the problem of medicament adhesion or sticking to the metering mechanism is alleviated or substantially reduced.

The amount of dry powder may be measured on a volume or weight basis.

Typically, the meter comprises a volume and/or a weight and/or time and/or surface area and/or a particle counting regulated mechanism.

In one embodiment, metering of medicament dose may be achievable by pulsing electrical current flow through the meter for a selected dispensing time.

For example, the meter may comprise a valve (for example, a linear or rotary valve) and/or a piston and/or a load cell. In another aspect, the dose-metering mechanism may comprise a plunger, such as might exist in a syringe. Embodiments including multiple plungers and multiple syringe chambers are also envisaged.

Preferably, the meter comprises at least one metering chamber. In one embodiment, on actuation of the meter, the or each metering chamber moves into fluid communication with the reservoir.

In one embodiment, the meter and the reservoir are relatively rotatable with respect to each other about a common central axis. Preferably, the or each metering chamber is adapted to be in fluid communication selectively with the reservoir or with the patient.

The or each metering chamber may have a variable volume. Alternatively, the or each metering chamber may have a fixed volume which is variable by insertion of a plunger or piston. The or each metering chamber may be formed from expandable material and/or have a telescopic or concertina arrangement.

In one embodiment, the inhaler further comprises a gas permeable dry powder retaining means below the or each metering chamber. The retaining means may be made from a gas-permeable filter, a mesh screen, a porous material or a perforated chamber element.

A reset mechanism may be provided for resetting the meter after actuation thereof. The reset means may for example, comprise a spring, motor, or other mechanical arrangement, and/or an electronic arrangement.

In a preferred aspect, the inhaler further comprises transport means to transport the metered volume from the reservoir to a delivery position. Preferably, the transport means is actuable by the meter.

Preferably, the inhaler further comprises dose-release means.

As used herein, the term “dose-release means” refers to the means for the making available of the dose for release to the patient, and the actual dispensing (whether passive or active) to the patient.

Preferably, the release means is actuable by the coupling means and/or the meter and/or the transport means.

Typically, the breath sensor and/or the meter and/or the transport means actuates the release means immediately after, or concurrent with, the actuation of the meter.

In this embodiment, the invention ensures that only after a dose has been metered from the dry powder reservoir can the medicament be made available for inhalation by the patient. Accordingly, the metered dose does not remain waiting in a metering chamber or delivery unit or release chamber for any length of time and therefore there is substantially reduced or alleviated the chance of deposition or sticking of the medicament onto the walls of the device, or the chance of moisture ingress or contamination from the external environment.

The release may be active in the sense that medicament is actively dispensed from the container, or the release may be passive in the sense that medicament is merely made available for inhalation when the release means is actuated.

Therefore, the dose-release means may comprise (i) a passive and/or (ii) an active dose-release means.

In one embodiment, the release means is passive and comprises making the metered dose available to the patient for inhalation thereby.

In another embodiment, the release means is active and comprises means to propel pressurised gas in the direction of patient inhalation.

In this embodiment, the patient receives a positive signal that the dose has been dispensed which may add to patient confidence. An active release means may also increase the efficacy of delivery of the medicament, for example, the drug may be released in a more focussed plume or cloud towards the back of the inhaler's nose or throat.

Preferably, the gas-propelling means provides at least one pulse of gas on actuation.

The gas-propelling means may provide one pulse of gas for each dose dispensed.

The gas may be air or an inert gas.

In another embodiment, the inhaler additionally comprises climate control means. Preferably, the climate control means is actuable by the coupling means and/or the meter and/or the transport means and/or the release means.

The climate control means may comprise means to (i) reduce moisture increase in the inhaler; and/or (ii) maintain ambient temperature; and/or (iii) dry the meter prior to actuation of the inhaler.

The climate control means may comprise a desiccant and/or a heater.

The heater may dry the meter prior to metering of the dose and/or immediately after the dose is dispensed.

The climate control means may comprise a temperature and/or a moisture sensor.

The coupling means may comprise a spring and/or a lever. Alternatively, or in addition, the coupling means may comprise a solenoid.

In one embodiment, the coupling means is reversibly deformable in response to heating thereof or application of a magnetic field thereto.

The inhaler may additionally comprise a reset coupling which is reversibly deformable in response to heating thereof or application of a magnetic field thereto.

Preferably, heating is achievable by electric current flow through the coupling or reset coupling.

Preferably, the coupling or reset coupling comprises a wire, strip, coil or tube.

Arrangements comprising multiple strips, wires, coils, or tubes are also envisaged. The multiple strips, wires, coils, or tubes may be arranged in any suitable fashion including parallel or series arrangements and bundle arrangements.

In one particular aspect, the coupling or reset coupling comprises one or more wires which contract in response to heating or application of a magnetic field thereto.

Preferably, the degree of contraction of the coupling is from 2% to 8%.

In one embodiment, the coupling comprises an alloy which undergoes a phase transition on heating (shape memory alloys). Certain shape memory alloys also undergo a change in shape on re-cooling. Such two way shape memory alloys are also envisaged for use herein.

In one embodiment, the shape memory alloy is preferably a nickel-titanium alloy such as a nickel-titanium alloy comprising from 5% to 95%, preferably from 20% to 80%, nickel by weight and from 95% to 5%, preferably from 80% to 20%, titanium by weight. By nickel-titanium alloy it is meant an alloy comprised essentially of nickel and titanium, although other elements such as Cu and Nb may be present in small (e.g. trace) amounts.

In other embodiments, the shape memory alloy is preferably a copper-aluminium-nickel alloy or a copper-zinc-aluminium alloy. Trace amounts of other elements may also be present.

In further embodiments, the coupling comprises an alloy which undergoes a phase transition on application of a magnetic field thereto (magnetic shape memory alloys). These materials are generally intermetallic, ferromagnetic alloys that exhibit twin variants in the martensitic, or low-temperature, phase of the material. Suitable magnetic shape memory alloys are for example, described in U.S. Pat. No. 5,958,154.

In one embodiment, the magnetic shape memory alloy exhibits an austenitic crystal structure above a characteristic phase transformation temperature and also exhibits a martensitic twinned crystal structure below the phase transformation temperature. The alloy has a magnetocrystalline anisotropy energy that is sufficient to enable motion of twin boundaries of the martensitic twinned crystal structure in response to application of a magnetic field to the martensitic twinned crystal structure.

Where a magnetic shape memory alloy is employed the inhaler preferably includes a magnetic field source disposed with respect to the coupling in an orientation that applies to the coupling a magnetic actuation field in a direction that is substantially parallel with a selected twin boundary direction of the martensitic twinned crystal structure of the coupling material.

Alternatively, the inhaler preferably includes a magnetic bias field source disposed with respect to the coupling in an orientation that applies a magnetic bias field to the coupling, and a magnetic actuation field source disposed with respect to the coupling in an orientation that applies a magnetic actuation field to the coupling material in a direction that is substantially perpendicular to the orientation of the applied magnetic bias field.

A preferred magnetic shape memory alloy is the actuator material comprising an alloy composition defined as Ni_{65-x-y}Mn₂₀+xGa₁₅+y, where x is between 3 atomic % and 15 atomic % and y is between 3 atomic % and 12 atomic %. Preferably, the actuator material comprises an alloy composition defined as Ni_{65-x-y}Mn₂₀+xGa₁₅+y, where x is between 6 atomic % and 10 atomic % and y is between 5 atomic % and 9 atomic %; or where x is between 12 atomic % and 15 atomic % and y is between 3 atomic % and 6 atomic %; or where x is between 10 atomic % and 14 atomic % and y is between 3 atomic % and 6 atomic %; or where x is between 7 atomic % and 11 atomic % and y is between 3 atomic % and 7 atomic %. In a particularly preferred aspect, the alloy is Ni₅₀Mn₂₅Ga_{25.}

Another preferred magnetic shape memory alloy is the alloy having the composition (NiₐFe_{b}Co_{c})_{65-x-y}(Mn_{d}FeₑCo_{f})₂₀+x(Ga_{g}SiₕAlᵢ)₁₅+y, where x is between 3 atomic % and 15 atomic % and y is between 3 atomic % and 12 atomic %, and where a+b+c=1, where d+e+f=1, and g+h+i=1.

In preferred aspects, b is between zero and 0.6, c is between zero and 0.6, and e, f, h and i are each zero; or b and c are each zero, e is between zero and 0.6, f is between zero and 0.6, and h and i are each zero; or b, c, e and f are each zero, h is between zero and 0.5, and i is between zero and 0.5.

Preferably, the one or more wires have a diameter from 30 to 400 micrometers, preferably from 50 to 150 micrometers.

Preferably, the coupling comprises from two to twelve, preferably six to ten wires which contract in response to heating or application of a magnetic field thereto.

The wires may be arranged in any suitable fashion including parallel or series arrangements and bundle arrangements.

In another aspect, the coupling comprises a strip which comprises multiple layers of different metals. Suitable strips typically comprise a plurality of layers of material, each material having a different coefficient of thermal expansion.

Preferred examples of strips include those comprising multiple layers of different metals (e.g. bimetallic strips) and strips comprising at least one piezoelectric material. Suitable piezoelectric materials include piezoelectric ceramics, such as compounds of lead zirconate and lead titanate, and piezoelectric crystals which are generally polycrystalline ferroelectric materials with the perovskite structure.

In one aspect, the coupling is deformable in response to heating arising from electrical current flow in the range from 0.01 A to 100 A, preferably from 0.1 A to 5 A. Alternatively, the coupling is deformable in response to heating arising from the application of an electrical voltage, particularly where the coupling comprises a piezoelectric material.

In another aspect, the coupling is deformable in response to a magnetic field of from 0.01 to 100 Tesla. The magnetic field may for example, be produced by a permanent magnet or by an electromagnet.

The deformation of the coupling (e.g. by electrical current flow therethrough) may be responsive to the detection of the inward breath of a patient. Alternatively, deformation of the coupling (e.g. by electrical current flow therethrough) may be responsive to a trigger coupled to any point in the breathing pattern of the patient, such as the end of the outward breath.

As used herein the term breath sensor encompasses any suitable means for monitoring, measuring, tracking or indicating the breath of a patient and may comprise one or more sensors.

Preferably, the breath sensor electro-mechanically actuates the meter at a predetermined trigger point in the patient's breath cycle. For example, the trigger point may be during the inhalation or exhalation stage of the patient's breath cycle.

In one aspect, the sensor comprises a breath-movable element which is movable in response to the breath of a patient. Preferably, the breath-movable element is selected from the group consisting of a vane, a sail, a piston, a diaphragm and an impeller.

Movement of the breath-movable element may be detectable by any suitable technique for detecting movement. Suitable techniques include optical detectors, magnetic detectors or detectors using detection of capacitative effects.

Optical detectors may be used to detect movement of the breath-movable element by providing the element with a patterned outer surface, for example strips in a barcode type arrangement, and locating the optical detector so that it points towards the patterned surface. Movement of the breath-movable element alters the amount of the light source which reflects back onto the optical detector as the beam passes over the patterned surface. The strips may be arranged so that the direction of movement of the element can be detected.

Magnetic detectors may be used to detect the movement of breath-movable element by the use of a magnetic switch device. A reader is located on the dispenser and magnetic material embedded within the breath-movable element (or vice-versa). Movement of the breath-movable element results in a change of the magnetic field experienced by the reader. Alternatively, a Hall effect device can be used whereby a semiconductor measures the strength of the magnetic field of the magnetic material on the breath-movable element.

Detection of capacitative effects may be used to detect movement of the breath-movable element by adding a conductive part to the element and also to a second fixed part of the dispenser. Movement of the breath-movable element results in a change in capacitance which can be measured.

In another aspect, the sensor comprises a pressure sensor for sensing the pressure profile associated with the breath of a patient. A pressure transducer is an example of a suitable pressure sensor.

In another aspect, the sensor comprises an airflow sensor for sensing the airflow profile associated with the breath of a patient.

In another aspect, the sensor comprises a temperature sensor for sensing the temperature profile associated with the breath of a patient.

In another aspect, the sensor comprises a moisture sensor for sensing the moisture profile associated with the breath of a patient.

In another aspect, the sensor comprises a gas sensor for sensing the chemical profile, for example, the oxygen or carbon dioxide profile associated with the breath of a patient.

Preferably, the sensor is connectable to an electronic information processor. The connection may be direct or via any suitable mechanical or electronic transfer means.

Preferably, the electronic information processor actuates the meter at a predetermined trigger point in the breath cycle.

Preferably, the electronic information processor includes an active memory for storing information about the breath cycle.

Suitably, the electronic information processor includes a predictive algorithm or look-up table for predicting the optimum trigger point. For example, a real-time analysis of the patient waveform may be made and the optimum trigger point derived by reference to that analysed waveform.

Suitably, the electronic information processor includes a second predictive algorithm or look-up table for predicting the optimum amount of medicament to release. Suitably, the electronic information processor includes a dose memory for storing information about earlier delivered doses and reference is made to the dose memory in predicting the optimum amount of medicament to release.

Preferably, the inhaler additionally comprises a display for displaying information about the optimum amount of medicament to release.

Preferably, the inhaler according additionally comprises a selector for selecting the amount of medicament to release.

In one aspect, the selector is manually operable.

Alternatively or in addition, the selector is operable in response to a signal from the electronic information processor.

Preferably, the selector comprises a timing mechanism for varying the time interval of actuation of the meter and/or dose-release mechanism.

The selector may comprise a multiple-fire mechanism for multiple actuation of the inhaler wherein each actuation releases a portion of the optimum amount of medicament.

Preferably, the inhaler additionally comprises an electrical energy source. In one aspect, the electrical energy source comprises a voltaic cell or battery of voltaic cells which may be rechargeable. In another aspect, the electrical energy source comprises a photovoltaic cell or battery of photovoltaic cells. The additional energy source may be mechanically-generated, for example, the energy source may comprise a biasable resilient member, e.g. a spring. Therefore, the electrical energy source may comprise a converter for converting mechanical energy into electrical energy.

The energy source may comprise a source of compressed fluid, preferably compressed gas, or a chemical energy store, preferably a chemical propellant or ignition mixture. Other sources may include physical explosives such as liquefied or solidified gas in a canister which burst when heated or exposed to the atmosphere.

Any electrical circuit may incorporate voltage amplification means for generating a higher voltage than that supplied by the voltaic cell or battery of voltaic cells, for example a step-up or inverting switching circuit or a dc-dc converter incorporating an oscillator, transformer and rectifier.

The electrical circuit may incorporate one or more energy storage components such as capacitors or inductors in order to supply a high enough instantaneous current to raise the temperature of the strips or wires at the required rate to the required temperature.

The input to the electrical circuit may be connected to the electrical energy source by means of a mechanical, electromechanical or electronic switching component.

The output of the electrical circuit may be connected to the strips or wires or to an electromagnet by means of a mechanical, electromechanical or electronic switching component or by a component allowing the output current to be controlled in a linear or digital (e.g. pulse width modulated) manner.

The strip or wire components may be powered from the battery using a switching component without additional power supply circuitry.

Suitably, the inhaler additionally comprises a controller for controlling the amount of electrical current flow through the coupling or to an electromagnet.

Suitably, the inhaler additionally comprises a timer for controlling the duration of electrical current flow through the coupling or to an electromagnet.

Suitably, the inhaler additionally comprises a local electrical store such as a capacitor or inductor.

Suitably, the inhaler is provided with a manual override to enable actuation of the device in the event of loss of electrical power. For example in the event of an emergency or system failure.

Preferably, the inhaler includes a safety mechanism to prevent unintended multiple actuations of the device. The patient is thereby protected from inadvertently receiving multiple doses of medicament in a situation where they take a number of short rapid breaths. More preferably, the safety mechanism imposes a time delay between successive actuations of the device. The time delay is typically in the order of from three to thirty seconds.

Preferably the inhaler comprises an actuation or dose counter for counting the number of actuations of the meter or dose-release means or releases of dose therefrom. More preferably, counting will occur even if the metering and/or release means is manually actuated, that is, the actuation counter is independent of the coupling between the breath sensor and the dose-dispensing means.

The actuation counter may be mechanical or electronic.

Suitably, the inhaler is provided with child-resistance features to prevent undesirable actuation thereof by a young child.

The inhaler of the invention is suitable for dispensing medicament, particularly for the treatment of respiratory disorders such as asthma and chronic obstructive pulmonary disease (COPD).

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate, ketotifen or nedocromil; antiinfectives e.g., cephalosporins, penicillins, streptomycin, sulphonamides, tetracyclines and pentamidine; antihistamines, e.g., methapyrilene; anti-inflammatories, e.g., beclomethasone dipropionate, fluticasone propionate, flunisolide, budesonide, rofleponide, mometasone furoate or triamcinolone acetonide; antitussives, e.g., noscapine; bronchodilators, e.g., albuterol, saimeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, or (−)-4-amino-3,5-dichloro-α-[[[6-[2-(2-pyridinyl)ethoxy]hexyl]methyl ]benzenemethanol; diuretics, e.g., amiloride; anticholinergics, e.g., ipratropium, tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Medicaments can also be delivered in combinations. Preferred formulations containing combinations of active ingredients contain salbutamol (e.g., as the free base or the sulphate salt) or salmeterol (e.g., as the xinafoate salt) in combination with an antiinflammatory steroid such as a beclomethasone ester (e.g., the dipropionate) or a fluticasone ester (e.g., the propionate). A particularly preferred combination comprises salmeterol xinafoate salt and fluticasone propionate.

Preferred medicaments are selected from albuterol, salmeterol, fluticasone propionate and beclomethasone dipropionate and salts or solvates thereof, e.g., the sulphate of albuterol and the xinafoate of salmeterol, and any mixtures thereof. Alternatively, the dispenser may be employed for dispensing vaccine.

Indeed, it is envisioned in accordance with this invention that any suitable diagnostic, prophylactic or therapeutic agent can used with the inhaler herein. Generally, drug particles suitable for delivery to the bronchial or alveolar region of the lung have an aerodynamic diameter of less than 10 micrometers. Other sized particles may be used if delivery to other portions of the respiratory tract is desired, such as the nasal cavity, mouth or throat. The medicament may be a pure drug, but more appropriately, it is preferred that powder comprise a drug mixed with a bulking agent (excipient), for example, lactose.

Additional powders may be engineered with particular densities, size ranges, or characteristics. Particles may comprise active agents, surfactants, wall forming materials, or other components considered desirable by those of ordinary skill.

Blends of bulking agents and drugs are typically formulated to allow the precise metering and dispersion on the powder into doses. A standard blend, for example, contains 13000 micrograms lactose mixed with 50 micrograms drug, yielding an excipient to drug ratio of 260:1. Because the present invention can meter and dispense such blends more accurately and effectively, dosage blends with excipient to drug ratios of 60:1, and potentially 2:1, may be used. At very low blend levels, however, the drug dose reproducibility becomes more variable Typically, the dry powder medicament includes a pharmaceutical excipient in dry powder form.

In one embodiment, the density of the dry powder medicament particles is reduced relative to standard dry powder medicament.

In another embodiment, the dry powder medicament particles are aerodynamically shaped to improve medicament delivery to the patient.

According to another aspect of the present invention there is provided an actuator for a dry powder medicament container having a meter for metering a volume of medicament, the actuator comprising a dispenser seat for receipt of the meter, a breath sensor, and an electromechanical coupling means for actuating the meter, wherein the coupling means is responsive to the breath sensor.

In one embodiment, the coupling means is reversibly deformable in response to heating thereof or application of a magnetic field thereto.

In another aspect, the invention provides a dry powder medicament container having a meter for use in the inhaler or the actuator as described hereinabove.

In still a further aspect, the invention provides a kit of parts comprising an inhaler as described hereinabove in the form of a cartridge; and a housing shaped for receipt of the cartridge.

In yet another aspect, the invention provides method for the delivery of an inhalable dry powder medicament to a patient, comprising:

(i) sensing the breath of a patient by use of a breath sensor;

(ii) at a trigger point, sending an actuation signal from the breath sensor to a meter for metering a volume of medicament from a medicament reservoir; and

(iv) releasing the inhalable medicament for inhalation by the patient, wherein the breath sensor electro-mechanically actuates the meter immediately prior to or concurrent with release of the medicament to the patient.

Preferably, the method further comprises the actuation of transport means to separate the metered volume from the reservoir, and/or dose-release means to release the dose for inhalation by the patient.

### BRIEF DESCRIPTION OF THE DRAWINGS

The invention will now be described further with reference to the accompanying figures in which:

FIG. 1 shows a typical patient inhalation profile of airflow (litres per minute) against time (seconds) as a patient inhales using a medicament dispenser according to the invention;

FIG. 2 shows a flow diagram of the sequence of events during the dispensing of a dose of medicament to a patient, wherein the inhaler includes a heater to dry the meter apparatus according to one aspect of the invention;

FIG. 3 shows a metering mechanism for a medicament dispenser according to one aspect of the invention wherein metering is by volume;

FIG. 4 shows a metering mechanism for a medicament dispenser according to another aspect of the invention wherein metering is by weight;

FIG. 5 shows a metering mechanism for a medicament dispenser according to another aspect of the invention wherein metering is by surface area;

FIG. 6 shows a metering mechanism for a medicament dispenser according to another aspect of the invention wherein metering is by time; and

FIG. 7 shows a medicament dispenser having a metering mechanism as shown in FIG. 3 and an associated system diagram linking the transport means to an electromechanical coupling according to one aspect of the invention.

### DETAILED DESCRIPTION OF THE DRAWINGS

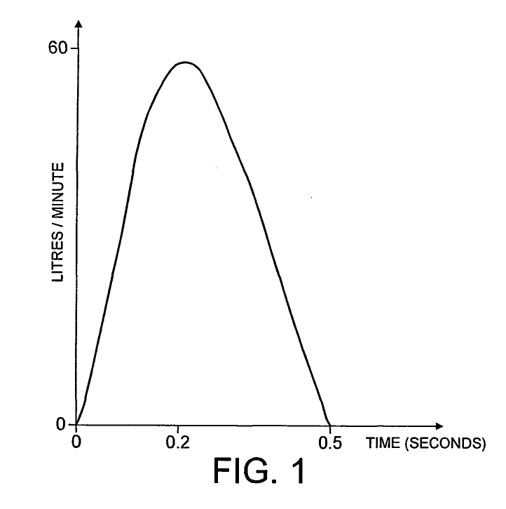

Referring now to the figures, a typical inhalation profile of an adult patient is illustrated in FIG. 1. In this example, the maximum airflow is 60 litres min⁻¹, approximately 200 ms after the breath is initiated. Optimally, a medicament dispenser responsive to a breath sensor should complete the dispensing cycle and make available the medicament for inhalation before the end of the patient's breath cycle.

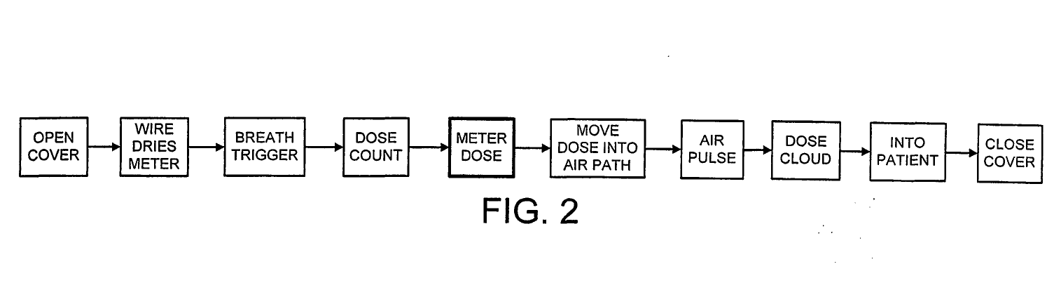

FIG. 2 illustrates the sequence of events during the use of a medicament dispenser in the form of a dry powder inhaler. If the inhaler comprises a protective cover, the cover is removed to expose a mouthpiece. Opening the protective cover activates a heating element to dry a metering pocket in the inhaler and thus alleviate any problems associated with condensation or general environmental moisture. After the patient starts to inhale, a breath sensor is activated and a flow sensor actuates the metering of the medicament from a medicament reservoir at a threshold airflow value. A transport means then transports the dose from a non-dispensing position to a delivery position wherein the medicament is ready for inhalation by the patient. Aerosolisation means in the form of an air pulse generator produces a dose cloud directed towards the patient through the mouthpiece so that the patient may sense the dose entering the mouth and thus the target airways. Notably, the patient has not had to provide any manual intervention throughout the entire metering and dispensing sequence.

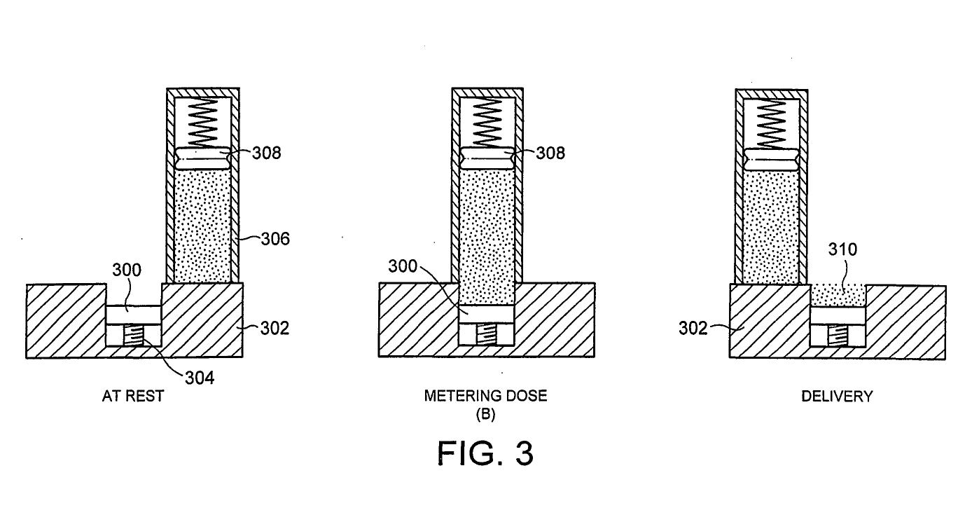

FIG. 3 illustrates a volumetric metering mechanism for use inside a medicament dispenser according to one aspect of the invention. A metering plate 300 is positioned in a dose plate 302 and is sited on an adjustable screw 304 for altering the volume of dose to be metered as required. The adjustable screw 304 may be driven by an ultrasonic motor (not shown). A medicament reservoir 306 is movable relative to the dose plate 302. In the metering position (B) the reservoir 306 is positioned directly over the metering plate 300 and the medicament 310 is dispensed onto the metering plate 300. Metering may occur by gravitational force. However, in this case a spring loaded plunger 308 is sited in the medicament reservoir 306 to maintain a constant pressure on the medicament throughout the life of the device.

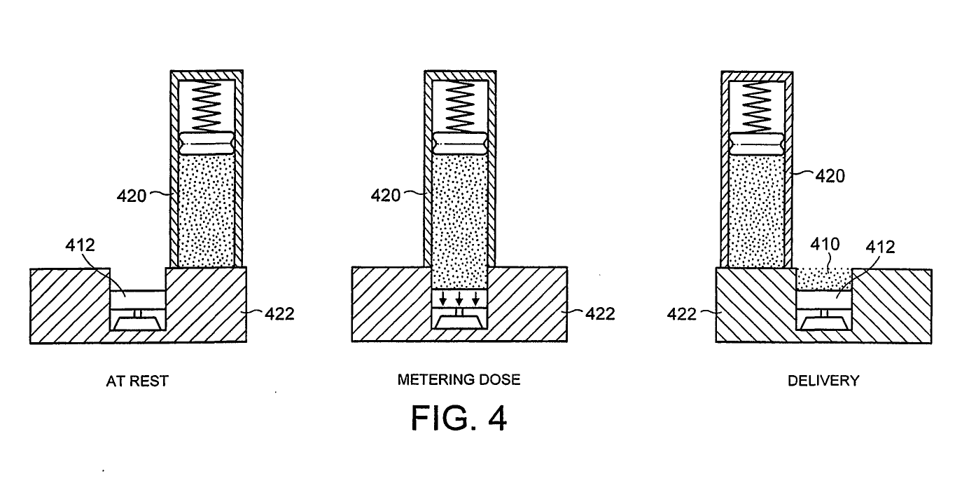

Metering may be by weight as shown in FIG. 4. In this example, the metering plate of FIG. 3 is replaced by a load cell 412 which measures the amount of medicament on its surface. Once the required weight of medicament 410 is dispensed from the reservoir 420, the reservoir 420 and/or the dose plate 422 are moved relative to each other to transport the metered dose to a delivery position.

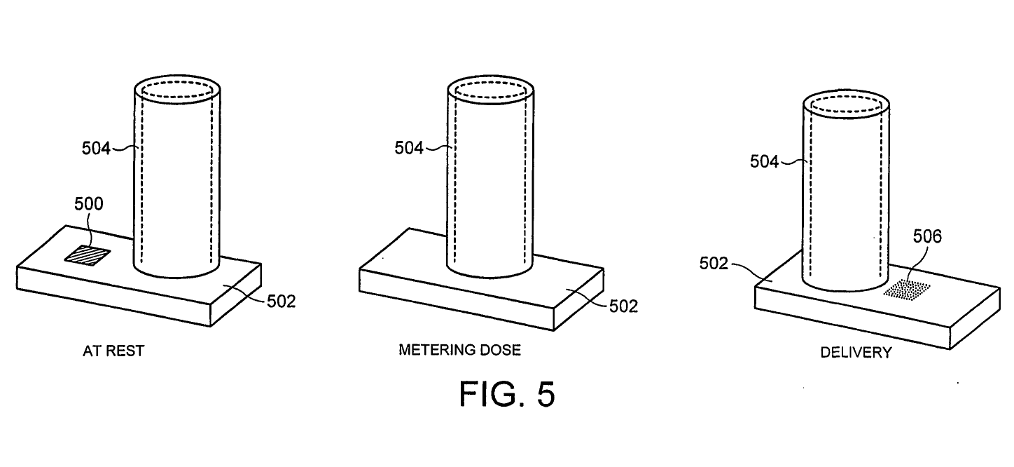

Metering may be by surface area as illustrated in FIG. 5. In this case, a defined metering area 500 is sited on the dose plate 502. Prior to metering of the medicament, the metering plate 500 is electrostatically charged so that it attracts medicament particles. After actuation of the metering mechanism, the metering area 500 is moved into direct communication with the medicament reservoir 504. Medicament particles 506 within the medicament reservoir 504 are electrostatically charged with an opposite polarity to the metering area 500. Thus, a monolayer of particles 506 is attracted to the area 500. Movement of the reservoir 504 away from the metering area 500 reveals a metered dose of particles 506. In one embodiment, after metering, the polarity of the particles in the reservoir 504 or the area 500 may be reversed so as to stop further attraction.

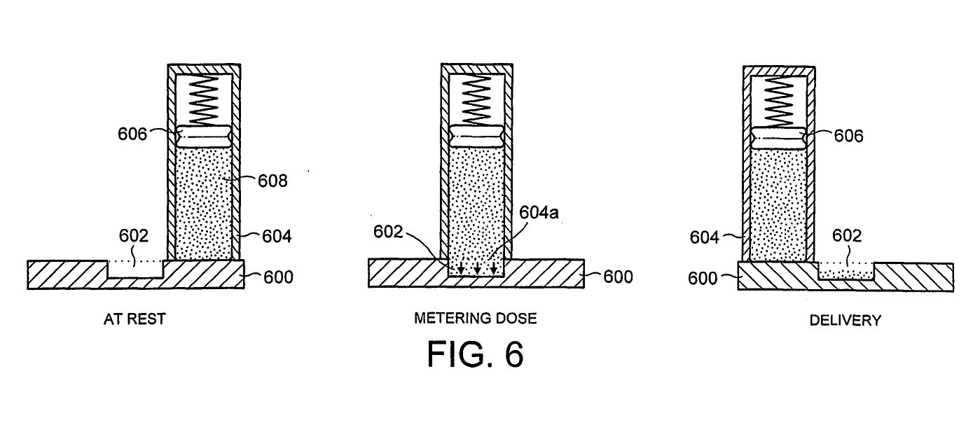

FIG. 6 illustrates a metering mechanism using time. A dose plate 600 has a metering cavity 602. A medicament reservoir 604 contains a spring-tensioned plunger 606 to maintain a constant pressure on the contents 608 of the medicament reservoir 604. In order to meter the medicament, the dose plate 600 is moved relative to the open end 604a of the reservoir 604 at a defined speed so that the medicament flows into the cavity 602 at a constant flow rate. The amount of metered medicament will be measured as a function of the time the cavity 602 is exposed to the reservoir outlet 604a.

Metering may also be achieved by particle counting. In this case, the particles are optically counted by lasers as they flow into the cavity 602. The reservoir 604 is moved away from the cavity 602 after a predetermined number of medicament particles has flowed therein.

In another embodiment, threaded screw means may be used to meter the medicament.

Although the FIGS. 3 to 6 show only one metering chamber, it can be envisaged that there may be a plurality of metering chambers and further, actuation of the inhaler may result in the metering and transporting of medicament in more than one chamber to the patient. In this case the inhaler will further comprise a dose controlling means. Thus the invention is relevant to unit dose inhalers, single-dose inhalers, and multi-dose inhalers.

The inhaler may also comprise a heater (not shown), in the form of a wire, to dry a dose plate prior to metering a dose of medicament. This has the advantage of removing any moisture from the dose plate that might adversely affect the metering of a dose. The heater may be triggered either immediately prior to metering a dose, immediately post metering a dose, or immediately after a dose has been dispensed.

Movement of, inter alia, the dose plate and/or the medicament reservoir, may be driven by a DC motor, a piezo-electric motor (e.g. an ultrasonic motor), and/or shape memory alloy (SMA) wires. For example, a DC motor may transform electrical energy from a power supply actuated by the patient's breath, to rotary and/or linear motion through gearing means and/or rack and pinion means. A piezo-electric motor (e.g. an ultrasonic motor) may drive rotary and/or linear motion. SMA wires may drive linear motion as activation of an electrical current through the wires causes the wires to contract in length.

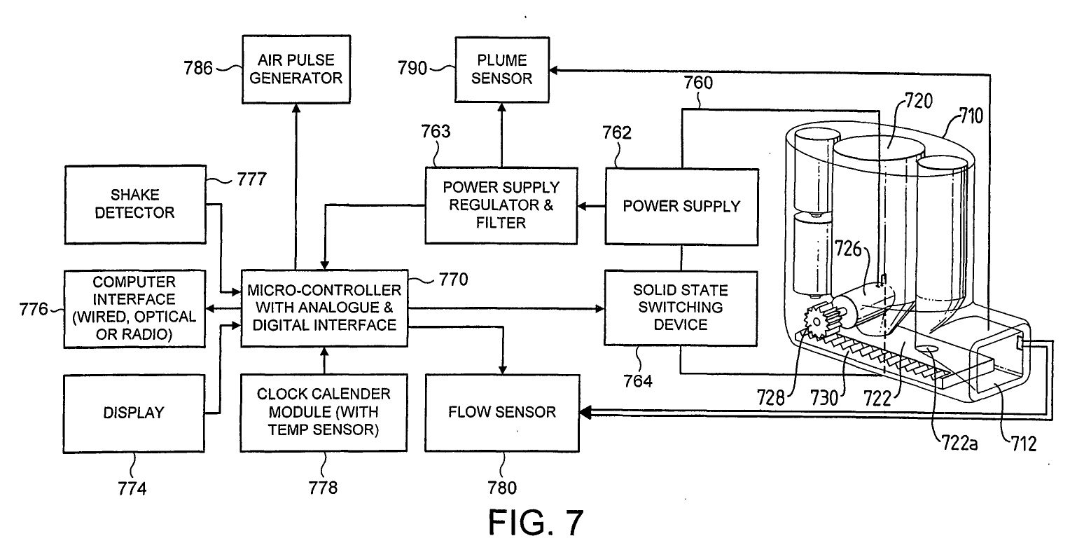

FIG. 7 shows a schematic representation of a breath-operable medicament dispensing system. The system comprises a metered dose inhaler similar according to the invention comprising a tubular housing 710 having a dispensing outlet 712 in the form of a mouthpiece. Within the housing 710 sits a medicament reservoir which has a volumetric dose-metering mechanism as illustrated in FIG. 3. A slide plate 722 is transportable between a metering position X and a delivery position Y enabling the passage of dispensed dose in a medicament pocket 722a to the dispensing outlet 712.

A DC motor 726 drives a rotary gear wheel 728 which in turn drives the slide plate 722 by the rack 730. Control of electrical current flow to the DC motor 726 is achievable using the illustrated circuitry. The DC motor 726 is connected to actuation circuit 760 which includes a power supply 762 (e.g. a voltaic cell or battery of voltaic cells) and a switch 764 in the form of a solid state switching device. The switch 764 itself connects to control circuitry including micro-controller 770 which has an analogue and/or digital interface. The power supply for the control circuitry is taken from the power supply 762 after suitable regulation and filtering 763. The micro-controller 770 itself connects with a flow sensor 780 which is associated with a breath sensor 790.

It may be appreciated that current flow to the DC motor 726 and hence actuation of the transport means 722 may be achievable as follows. The patient inhales through the mouthpiece 724 resulting in a change in airflow within the housing 710. The change in airflow is detected by the flow sensor 780 which sends a signal to the micro-controller 770. The micro-controller 770, in turn sends a switching signal to the solid state switching device 764 which results in closing of the actuation circuit and electrical current flow therethrough. DC motor 726 thus drives the slide plate 722 to a metering position and then to a delivery position and hence, dispensing of the medicament to the inhaling patient.

It may also be seen in FIG. 7 that the micro-controller 770 is connected to a display 774 for display of information to the patient and also with a computer interface 776 for exchange of data therewith. Communication with the computer interface 776 may be via a wired, optical or radio communications link. The micro-controller 770 is may also be connected to shake detector 777 for use in detecting whether the container 720 is shaken prior to actuation of the transport means 722 and to a clock-calendar module 778 including a temperature sensor. All circuitry and components thereof including the power supply 762, display 774, shake detector 777, computer interface 776 and clock-calendar module 778 may be arranged to be present on the housing 710 such that the system is in the form of a discrete, hand-held device.

In addition, the micro-controller 770 is linked to an air pulse generator 786 for actuating the release mechanism for aerosolisation of the dose. The power supply 763 is connected to a plume sensor 790 which senses when a dose of medicament leaves the dispenser and feeds back to turn off the power supply.

It may be appreciated that any of the parts of the inhaler or actuator which contact the medicament suspension may be coated with materials such as fluoropolymer materials which reduce the tendency of medicament to adhere thereto. Any movable parts may also have coatings applied thereto which enhance their desired movement characteristics. Frictional coatings may therefore be applied to enhance frictional contact and lubricants used to reduce frictional contact as necessary.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. An inhaler for delivery of a dry powder medicament, the inhaler comprising: a breath sensor for sensing the breath of a patient; a reservoir for said dry powder; a meter for metering an amount of dry powder from said reservoir; and electromechanical coupling means for actuating said meter, wherein said coupling means is directly or indirectly responsive to said breath sensor.

2. An inhaler according to claim 1 wherein the meter comprises a volume and/or a weight and/or a time and/or a surface-area and/or a particle counting regulated mechanism.

3. An inhaler according to claim 1 or claim 2 wherein the meter comprises a valve (for example, a linear or rotary valve) and/or a piston and/or a load cell and/or a plunger.

4. An inhaler according to any one of the preceding claims wherein the meter comprises at least one metering chamber.

5. An inhaler according to claim 4 wherein on actuation of the meter, the or each metering chamber moves into fluid communication with the reservoir.

6. An inhaler according to claim 4 wherein the meter and the reservoir are relatively rotatable with respect to each other about a common central axis.

7. An inhaler according to claim 6 wherein the or each metering chamber is adapted to be in fluid communication selectively with the reservoir or with the patient.

8. An inhaler according to any one of claims 4 to 7 wherein the or each metering chamber has a variable volume.

9. An inhaler according to any one of claims 4 to 7 wherein the or each metering chamber has a fixed volume which metering volume is variable by insertion of a plunger or piston.

10. An inhaler according to any one of claims 4 to 9 wherein the or each metering chamber is formed from expandable material.

11. An inhaler according to any one of claims 4 to 9 wherein the or each metering chamber has a telescopic or concertina arrangement.

12. An inhaler according to any one of claims 4 to 11 further comprising a gas permeable dry powder retaining means below the or each metering chamber.

13. An inhaler according to claim 12 wherein the retaining means is made from a gas-permeable filter, a mesh screen, a porous material or a perforated chamber element.

14. An inhaler according to any one of the preceding claims, additionally comprising a reset mechanism for resetting the meter after actuation thereof.

15. An inhaler according to any one of the preceding claims further comprising transport means to transport the metered volume from the reservoir to a delivery position.

16. An inhaler according to claim 15 wherein the transport means is actuable by the meter.

17. An inhaler according to any one of the preceding claims further comprising dose-release means.

18. An inhaler according to claim 17 wherein the release means is actuable by the coupling means and/or the meter and/or the transport means.

19. An inhaler according to claim 17 or claim 18 wherein the dose-release means comprises (i) a passive and/or (ii) an active dose-release mechanism.

20. An inhaler according to any one of claims 17 to 19 wherein the dose-release mechanism is passive and comprises making the metered volume available to the patient for inhalation thereby.

21. An inhaler according to any one of claims 17 to 19 wherein the dose-release mechanism is active and comprises means to propel pressurised gas in the direction of patient inhalation.

22. An inhaler according to claim 21 wherein the gas-propelling means provides at least one pulse of gas on actuation.

23. An inhaler according to claim 21 or 22 wherein the gas-propelling means provides one pulse of gas for each dose dispensed.

24. An inhaler according to any one of claims 21 to 22 wherein the gas is air.

25. An inhaler according to any one of claims 21 to 23 wherein the gas is an inert gas.

26. An inhaler according to any one of the preceding claims additionally comprising climate control means.

27. An inhaler according to claim 26 wherein the climate control means is actuable by the coupling means and/or the meter and/or the transport means and/or the release means.

28. An inhaler according to claim 27 wherein the climate control means comprises means to (i) reduce moisture increase in the inhaler; and/or (ii) maintain ambient temperature; and/or (iii) dry the meter prior to actuation of the inhaler.

29. An inhaler according to any one of claims 26 to 28 wherein the climate control means comprises a desiccant.

30. An inhaler according to any one of claims 26 to 29 wherein the climate control means comprises a heater.

31. An inhaler according to claim 30 wherein the heater dries the meter prior to metering of the volume and/or immediately after the volume is released.

32. An inhaler according to any one of claims 26 to 31 wherein the climate control means comprises a temperature and/or a moisture sensor.

33. An inhaler according to any one of the preceding claims wherein the coupling means comprises a spring and/or a lever.

34. An inhaler according to any one of the preceding claims wherein the coupling means comprises a solenoid.

35. An inhaler as claimed in any one of the preceding claims wherein the coupling means is reversibly deformable in response to heating thereof or application of a magnetic field thereto.

36. An inhaler according to claim 35, additionally comprising a reset coupling which is reversibly deformable in response to heating thereof or application of a magnetic field thereto.

37. An inhaler according to claim 35 or 36, wherein the heating is achievable by electric current flow through the coupling.

38. An inhaler according to any of claims 35 to 37, wherein the coupling comprises a wire, strip, coil or tube.

39. An inhaler according to claim 38, wherein the coupling comprises multiple wires, strips, coils or tubes.

40. An inhaler according to any of claims 35 to 39, wherein the coupling comprises one or more wires which contract in response to heating or application of a magnetic field thereto.

41. An inhaler according to claim 40, wherein the coupling exhibits a degree of contraction of from 2% to 8% on heating or application of a magnetic field thereto.

42. An inhaler according to claim 41, wherein the coupling comprises an alloy which undergoes a phase transition on heating or application of a magnetic field thereto.

43. An inhaler according to claim 42, wherein the alloy is a nickel-titanium alloy.

44. An inhaler according to claim 43, wherein said nickel-titanium alloy comprises from 5% to 95% nickel by weight and from 95% to 5% titanium by weight, preferably from 20% to 80% nickel by weight and from 80% to 20% titanium by weight.

45. An inhaler according to either of claims 43 or 44, wherein the nickel-titanium alloy additionally comprises copper, niobium or any mixtures thereof.

46. An inhaler according to claim 42, wherein the alloy is a copper-zinc-aluminium alloy or a copper-aluminium-nickel alloy.

47. An inhaler according to claim 42, wherein the alloy has the composition defined as Ni_{65-x-y}Mn₂₀+xGa₁₅+y, where x is between 3 atomic % and 15 atomic % and y is between 3 atomic % and 12 atomic %.

48. An inhaler according to claim 42, wherein the alloy has the composition defined as (NiₐFe_{b}Co_{c})_{65-x-y}(Mn_{d}FeₑCo_{f})₂₀+x(Ga_{g}SiₕAlᵢ)₁₅+y, where x is between 3 atomic % and 15 atomic % and y is between 3 atomic % and 12 atomic %, and where a+b+c=1, where d+e+f=1, and g+h+i=1.

49. An inhaler according to any of claims 38 to 48, wherein the one or more wires have a diameter from 30 to 400 micrometers, preferably from 50 to 150 micrometers.

50. An inhaler according to any of 38 to 49, wherein the coupling comprises from two to twelve, preferably six to ten wires which contract in response to heating or application of a magnetic field thereto.

51. An inhaler according to claim 38 or 39, wherein said strip comprises multiple layers of different metals.

52. An inhaler according to claim 51, wherein the strip comprises a bimetallic strip.

53. An inhaler according to either of claims 51 or 52, wherein the strip comprises at least one piezoelectric material.

54. An inhaler according to any of claims 35 to 53, wherein the coupling is deformable in response to heating arising from electrical current flow in the range from 0.01 A to 100 A, preferably from 0.1 A to 5 A.

55. An inhaler according to any of claims 35 to 53, wherein the coupling is deformable in response to a magnetic field of from 0.01 to 100 Tesla.

56. An inhaler according to any one of the preceding claims wherein the breath sensor electro-mechanically actuates the meter at a predetermined trigger point in the patient's breath cycle.

57. An inhaler according to claim 56 wherein the trigger point is during the inhalation or exhalation stage of the patient's breath cycle.

58. An inhaler according to any one of the preceding claims wherein the breath sensor comprises a breath-movable element which is movable in response to the breath of a patient.

59. An inhaler according to claim 58, wherein the breath-movable element is selected from the group consisting of a vane, a sail, a piston, a diaphragm and an impeller.

60. An inhaler according to any one of the preceding claims wherein the sensor comprises a pressure sensor for sensing the pressure profile associated with the breath of a patient.

61. An inhaler according to any one of the preceding claims wherein the sensor comprises an airflow sensor for sensing the airflow profile associated with the breath of a patient.

62. An inhaler according to any one of the preceding claims wherein the sensor comprises a temperature sensor for sensing the temperature profile associated with the breath of a patient.

63. An inhaler according to any one of the preceding claims wherein the sensor comprises a moisture sensor for sensing the moisture profile associated with the breath of a patient.

64. An inhaler according to any one of the preceding claims wherein the sensor comprises a gas sensor for sensing the chemical profile, for example, the oxygen or carbon dioxide profile associated with the breath of a patient.

65. An inhaler according to any one of the preceding claims wherein the sensor is connectable to an electronic information processor.

66. An inhaler according to claim 65 wherein the electronic information processor actuates the meter at a predetermined trigger point in the breath cycle.

67. An inhaler as claimed in claim 65 or claim 66 wherein the electronic information processor includes an active memory for storing information about the breath cycle.

68. An inhaler according to claim 65 wherein the electronic information processor includes a predictive algorithm for predicting the optimum trigger point.

69. An inhaler according to claim 65 wherein the electronic information processor includes a look up table for predicting the optimum trigger point.

70. An inhaler according to any one of claims 67 to 69 wherein the electronic information processor includes a second predictive algorithm for predicting the optimum amount of medicament to release.

71. An inhaler according to any one of claims 67 to 69 wherein the electronic information processor includes a second look up table for predicting the optimum amount of medicament to release.

72. An inhaler according to claim 70 or 71 wherein the electronic information processor includes a dose memory for storing information about earlier delivered doses and reference is made to the dose memory in predicting the optimum amount of medicament to release.

73. An inhaler according to claims 70 to 72 additionally comprising a display for displaying information about the optimum amount of medicament to release.

74. An inhaler according to any one of claims 70 to 73 additionally comprising a selector for selecting the amount of medicament to release.

75. An inhaler according to claim 74 wherein the selector is manually operable.

76. An inhaler according to claim 74 wherein the selector is operable in response to a signal from the electronic information processor.

77. An inhaler according to claim 74 to 76 wherein the selector comprises a timing mechanism for varying the time interval of actuation of the dose-metering and/or dose-release means.

78. An inhaler according to any one of claims 74 to 77 wherein the selector comprises a multiple-fire mechanism for multiple actuation of the inhaler wherein each actuation releases a portion of the optimum amount of medicament.

79. An inhaler according to any of the preceding claims, additionally comprising an electrical energy source.

80. An inhaler according to claim 79, wherein the electrical energy source comprises a voltaic cell or battery of voltaic cells.

81. An inhaler according to claim 80, wherein the voltaic cell or battery of voltaic cells is rechargeable.

82. An inhaler according to claim 79, wherein the electrical energy source comprises a photovoltaic cell or battery of photovoltaic cells.

83. An inhaler according to claim 79, wherein the electrical energy source comprises a converter for converting mechanical energy into electrical energy.

84. An inhaler according to any of claims 79 to 83, additionally comprising a controller for controlling the amount of electrical current flow through the coupling or to an electromagnet.

85. An inhaler according to any of claims 79 to 84, additionally comprising a timer for controlling the duration of electrical current flow through the coupling or to an electromagnet.

86. An inhaler according to any of claims 79 to 85 additionally comprising a local electrical energy store.

87. An inhaler according to any one of claims 79 to 86 wherein the additional energy source is mechanically-generated.

88. An inhaler according to claim 87 wherein the energy source comprises a biasable resilient member.

89. An inhaler according to claim 88 wherein the biasable resilient member is a spring.

90. An inhaler according to claim 87 wherein the energy source comprises a source of compressed fluid, preferably compressed gas.

91. An inhaler according to claim 87 wherein the energy source comprises a chemical energy store, preferably a chemical propellant or ignition mixture.

92. An inhaler according to claim 87 wherein the energy source comprises a physically explosive energy source.

93. An inhaler according to any one of the preceding claims wherein the medicament is selected from the group consisting of albuterol, salmeterol, fluticasone propionate, beclomethasone dipropionate, salts or solvates thereof and any mixtures thereof.

94. An inhaler according to any one of the preceding claims wherein the dry powder medicament includes a pharmaceutical excipient in dry powder form.

95. An inhaler according to any one of the preceding claims wherein the density of the dry powder medicament particles is reduced relative to standard dry powder medicament.

96. An inhaler according to any one of the preceding claims wherein the dry powder medicament particles are aerodynamically shaped to improve medicament delivery to the patient.

97. An inhaler according to any one of the preceding claims comprising an actuation counter for counting the number of actuations of the meter and/or dose-releasing means or a dose counter for counting the number of doses delivered.

98. An inhaler according to claim 97, wherein the actuation counter is independent of the coupling between the breath sensor and the meter and/or dose-releasing means.

99. An inhaler according to any one of the preceding claims additionally comprising a safety mechanism to prevent unintended multiple actuations of the inhaler.

100. An inhaler according to any one of the preceding claims wherein the safety mechanism imposes a time delay between successive actuation of the inhaler.

101. An inhaler according to any of the preceding claims comprising a manual override enabling manual actuation of the dose-metering and/or dose-releasing means.

102. An inhaler according to claim 101 comprising a child resistance feature to prevent undesirable actuation thereof by children.

103. An actuator for use in an inhaler according to any one of the preceding claims.

104. An actuator for a dry powder medicament container having a meter for metering a volume of medicament, the actuator comprising a dispenser seat for receipt of the meter, a breath sensor, and an electromechanical coupling means for actuating the meter, wherein the coupling means is responsive to the breath sensor.

105. An actuator according to claim 104 wherein the coupling means is reversibly deformable in response to heating thereof or application of a magnetic field thereto.

106. A dry powder medicament container having a meter for use in the inhaler according to claims 1 to 102 and/or the actuator of claims 103 to 105.

107. Kit of parts comprising an inhaler according to any of claims 1 to 102 in the form of a cartridge; and a housing shaped for receipt of the cartridge.

108. A method for the delivery of an inhalable dry powder medicament to a patient, comprising:
(i) sensing the breath of a patient by use of a breath sensor;
(ii) at a trigger point, sending an actuation signal from the breath sensor to a meter for metering a volume of medicament from a medicament reservoir; and
(iv) releasing the inhalable medicament for inhalation by the patient, wherein the breath sensor electro-mechanically actuates the meter immediately prior to or concurrent with release of the medicament to the patient.

109. A method according to claim 108 further comprising the actuation of transport means to separate the metered volume from the reservoir and/or dose-release means to release the dose for inhalation by the patient.
